(19) United States
(12) Patent Application Publication
(10) Pub. No.: US 2018/0348191 A1
(43) Pub. Date: Dec. 6, 2018

(54) SYSTEM AND METHOD FOR IMPROVING FUEL QUALITY

(71) Applicant: General Electric Company, Schenectady, NY (US)

(72) Inventors: Raul Eduardo Ayala, Sugar Land, TX (US); George Frey, Houston, TX (US)

(21) Appl. No.: 15/608,844

(22) Filed: May 30, 2017

(51) Int. Cl. G01N 33/28; F02M 37/22; C10G 31/09; B01D 35/00; B01D 36/00

(52) U.S. Cl. CPC G01N 33/2847; F02M 37/221; C10G 31/09; B01D 36/003; B01D 36/005; G01N 33/28; F02M 37/22; B01D 35/005

(57) ABSTRACT

A system includes a gas turbine system that combusts a fuel to produce a power, and the gas turbine system is disposed in a gas turbine site. The system also includes an analyzer system that determines multiple batch fuel characteristics of a batch of the fuel, and the batch of the fuel is delivered via a transport system. Moreover, the system includes a small batch fuel processing system that receives the batch of fuel and filters the batch of fuel to a filtered batch of fuel based on the multiple fuel characteristics. Further, the filtered batch of fuel adheres to manufacturer fuel recommended characteristics for use in the gas turbine system.

### BACKGROUND

The subject matter disclosed herein relates to a system and method for improving, for example, the quality of fuel.

Industrial machines, such as gas turbine systems, may provide for the generation of power. For example, the gas turbine systems typically include a compressor for compressing a working fluid, such as air, a combustor for combusting the compressed working fluid with fuel, and a turbine for turning the combusted fluid into a rotative power. For example, the compressed air is injected into a combustor, which heats the fluid causing it to expand, and the expanded fluid is forced through the gas turbine. The gas turbine may then convert the expanded fluid into rotative power, for example, by a series of blade stages of the turbine. The rotative power may then be used to drive a load, which may include an electrical generator producing electrical power and electrically coupled to a power distribution grid. The fuel supplied for use in the combustor may vary in quality. It may be beneficial to improve the quality of the fuel for systems that may include gas turbine systems.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In a first embodiment, a system includes a gas turbine system that combusts a fuel to produce a power, and the gas turbine system is disposed in a gas turbine site. The system also includes an analyzer system that determines multiple batch fuel characteristics of a batch of the fuel, and the batch of the fuel is delivered via a transport system. Moreover, the system includes a small batch fuel processing system that receives the batch of fuel and filters the batch of fuel to a filtered batch of fuel based on the multiple fuel characteristics. Further, the filtered batch of fuel adheres to manufacturer fuel recommended characteristics for use in the gas turbine system.

In a second embodiment, a method includes receiving, via a transport system, a batch of fuel. The method further includes analyzing, via an analyzer system, the batch of fuel to derive multiple batch fuel characteristics. In addition, the method includes determining, via a processor, a suitability of the batch of fuel to be used in a gas turbine system based on the multiple batch fuel characteristics. Moreover, the method includes filtering, via a small batch fuel processing system, the batch of fuel to produce a filtered batch of fuel that adheres to manufacturer fuel recommended criteria for use in the gas turbine system when the batch of fuel is determined to be not suitable for use in the gas turbine system.

In a third embodiment, one or more tangible, non-transitory, machine-readable media including instructions that cause a processor to receive, via a transport system, a batch of fuel. The instructions also cause the processor to analyze, via an analyzer system, the batch of fuel to derive multiple batch fuel characteristics. Further, the instructions cause the processor to determine a suitability of the batch of fuel to be used in a gas turbine system based on the multiple batch fuel characteristics. Moreover, the instructions cause the processor to filter, via a small batch fuel processing system, the batch of fuel to produce a filtered batch of fuel that adheres to manufacturer fuel recommended criteria for use in the gas turbine system when the batch of fuel is determined to be not suitable for use in the gas turbine system.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a block diagram of an embodiment of a power production system having a gas turbine system;

FIG. 2 is a block diagram of an embodiment of a small batch fuel processing system;

FIG. 3 is a block diagram of an embodiment of a method to improve the quality of fuel received at a power production system; and

FIG. 4 is a detailed block diagram of an embodiment of a small batch fuel processing system.

### DETAILED DESCRIPTION

One or more specific embodiments of the present subject matter will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

A gas turbine system may operate using fuel with a particular range of characteristics. For example, some fuel may be within a manufacturer-specified range of characteristics (i.e., the fuel is “in spec”) and some fuel may be outside the manufacturer-specified range of characteristics (i.e., the fuel is “out of spec”). Some fuel that is considered out of spec may still be used in the gas turbine system, but may reduce the life of the gas turbine system.

Further, fuel may be delivered to the site of a gas turbine system by various methods (e.g., by trucks or by pipeline). The fuel that is delivered may sometimes be out of spec, and is thus not recommended for use in the gas turbine system. The out of spec fuel may be returned to the supplier, used for other purposes, or discarded. Certain properties of the out of spec fuel may be improved to change the out of spec fuel into in spec fuel that is within the recommended range of characteristics for use in the gas turbine system. The techniques described herein may allow for a system to improve the out of spec fuel so that it may become in spec fuel and thus more appropriate for use in the gas turbine system.

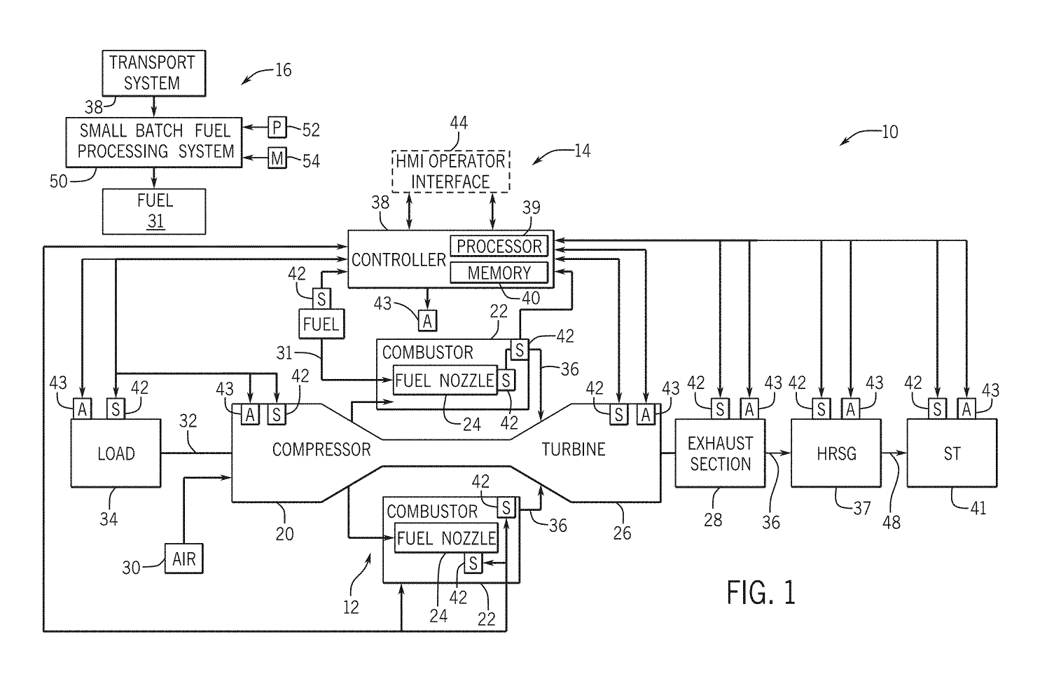

With the foregoing in mind, it may be useful to describe an embodiment of a turbomachinery incorporating techniques disclosed herein, such as a power production system 10 illustrated in FIG. 1. As illustrated in FIG. 1, the power production system 10 includes the gas turbine system 12, a monitoring and control system 14, and a fuel supply system 16. The gas turbine system 12 may include a compressor 20, combustion systems 22, fuel nozzles 24, a gas turbine 26, and an exhaust section 28. During operation, the gas turbine system 12 may pull air 30 into the compressor 20, which may then compress the air 30 and move the air 30 to the combustion system 22 (e.g., which may include a number of combustors). In the combustion system 22, the fuel nozzle 24 (or a number of fuel nozzles 24) may inject fuel that mixes with the compressed air 30 to create, for example, an air-fuel mixture.

The air-fuel mixture may combust in the combustion system 22 to generate hot combustion gases, which flow downstream into the turbine 26 to drive one or more turbine stages. For example, the combustion gases may move through the turbine 26 to drive one or more stages of turbine blades, which may in turn drive rotation of a shaft 32. The shaft 32 may connect to a load 34, such as a generator that uses the torque of the shaft 32 to produce electricity. After passing through the turbine 26, the hot combustion gases may vent as exhaust gases 36 into the environment by way of the exhaust section 28. The exhaust gas 36 may include gases such as carbon dioxide (CO₂), carbon monoxide (CO), nitrogen oxides (NOₓ), and so forth.

The exhaust gas 36 may include thermal energy, and the thermal energy may be recovered by a heat recovery steam generation (HRSG) system 37. In combined cycle systems, such as the power production system 10, hot exhaust 36 may flow from the gas turbine 26 and pass to the HRSG 37, where it may be used to generate high-pressure, high-temperature steam 48. The steam 48 produced by the HRSG 37 may then be passed through the steam turbine system 41 for further power generation. In addition, the produced steam may also be supplied to any other processes where steam may be used, such as to a gasifier used to combust the fuel to produce the untreated syngas. The gas turbine engine generation cycle is often referred to as the “topping cycle,” whereas the steam turbine engine generation cycle is often referred to as the “bottoming cycle.” Combining these two cycles may lead to greater efficiencies in both cycles. In particular, exhaust heat from the topping cycle may be captured and used to generate steam for use in the bottoming cycle.

In certain embodiments, the power production system 10 may also include a controller 38. The controller 38 may be communicatively coupled to a number of sensors 42 and one or more actuators 43 suitable for controlling components of the system 10. The actuators 43 may include valves, switches, positioners, pumps, and the like, suitable for controlling the various components of the system 10. The controller 38 may receive data from the sensors 42, and may be used to control the compressor 20, the combustors 22, the turbine 26, the exhaust section 28, the load 34, the HRSG 37, the steam turbine system 41, and so forth.

In certain embodiments, the sensors 42 may be any of various sensor types useful in providing various operational data to the controller 38. For example, the sensors 42 may provide flow, pressure, and temperature of the compressor 20, speed and temperature of the turbine 26, vibration of the compressor 20 and the turbine 26, as well as flow for the exhaust gas 36, temperature, pressure and emission (e.g., CO₂, NOₓ) levels in the exhaust gas 36, properties of a batch of fuel 31 (e.g., carbon content, Wobbe index, cetane number, octane number, and so on), temperature of the batch of fuel 31, temperature, pressure, clearance of the compressor 20 and the turbine 26 (e.g., distance between the rotating and stationary parts of the compressor 20, between the rotating and stationary parts of the turbine 26, and/or between other stationary and rotating components), flame temperature or intensity, vibration, combustion dynamics (e.g., fluctuations in pressure, flame intensity, and so forth), load data from load 34, output power from the turbine 26, and so forth. The sensors 42 may also include temperature sensors such as thermocouples, thermistors, and the like, disposed in the steam turbine system 41. The sensors 42 may also include flow sensors such as flowmeters (e.g., differential pressure flowmeters, velocity flowmeters, mass flowmeters, positive displacement flowmeters, open channel flowmeters) and liquid level sensors such as continuous level transmitters, ultrasonic transducers, laser level transmitters, and so on, disposed in the steam turbine system 41.

Additionally, the sensors 42 may include pressure sensors such as piezo-resistive pressure sensors, differential pressure sensors, optical pressure sensors, and so on, included in the steam turbine system 41. Fuel 31 properties may be sensed and/or otherwise provided to the controller 38, e.g., via a human operator interface 44. The fuel 31 properties may include moisture content, carbon content, chemical composition, specific gravity, ambient temperature, energy content, certain “numbers” (e.g., Wobbe Index, cetane number, octane number, and so on), or a combination thereof. In certain embodiments, the controller 38 may be communicatively coupled to a number of sensors 42, a human machine interface (HMI) operator interface 44, and one or more actuators 43 suitable for controlling components of the power production system 10. The actuators 43 may include valves, switches, positioners, pumps, and the like, suitable for controlling the various components of the power production system 10. The controller 38 may receive data from the sensors 42, and may be used to control the compressor 20, the combustors 22, the turbine 26, the exhaust section 28, the load 34, the HRSG 37, and so forth.

In certain embodiments, the HMI operator interface 44 may be executable by one or more computer systems of the power production system 10. A plant operator may interface with the power production system 10 via the HMI operator interface 44. Accordingly, the HMI operator interface 44 may include various input and output devices (e.g., mouse, keyboard, monitor, touch screen, or other suitable input and/or output device) such that the plant operator may provide commands (e.g., control and/or operational commands) to the controller 38.

The controller 38 may include a processor(s) 39 (e.g., a microprocessor(s)) that may execute software programs to control the power production system 10. Moreover, the processor 39 may include multiple microprocessors, one or more “general-purpose” microprocessors, one or more special-purpose microprocessors, and/or one or more application specific integrated circuits (ASICS), or some combination thereof. For example, the processor 39 may include one or more reduced instruction set (RISC) processors. The controller 38 may include a memory device 40 that may store information such as control software, look up tables, configuration data, etc.

The memory device 40 may include a tangible, non-transitory, machine-readable medium, such as a volatile memory (e.g., a random access memory (RAM)) and/or a nonvolatile memory (e.g., a read-only memory (ROM), flash memory, a hard drive, or any other suitable optical, magnetic, or solid-state storage medium, or a combination thereof). The memory device 40 may store a variety of information, which may be suitable for various purposes. For example, the memory device 40 may store machine-readable and/or processor-executable instructions (e.g., firmware or software) for the processor execution.

The power production system 10 also includes the fuel supply system 16 that provides the batch of fuel 31 through the fuel nozzles 24. A transport system 38 may transport the batch of fuel 31 (e.g., naphtha, kerosene, Arabian super light, or any other liquid fuel) to the location of the power production system 10. The transport system 38 may include a combination of pipelines and vehicles (e.g., trucks and boats) in order to transport the batch of fuel 31 to the location of the power production system 10. In some embodiments, the fuel supply system 16 may include a small batch fuel processing system 50 that may be utilized to improve the quality of the batch of fuel 31. After the batch of fuel 31 has arrived at the location of the power production system 10, the batch of fuel 31 may be analyzed. If the analysis shows that it may be beneficial to improve certain properties of the batch of fuel 31, the batch of fuel 31 may be processed by the small batch fuel processing system 50 to improve the batch of fuel 31, as further described below.

Further, the small batch fuel processing system 50 may include a processor 52 and a memory device 54. The processor 52 and memory device 54 may be part of the controller 38, or may be separate from the controller 38. Moreover, the processor 52 may include multiple microprocessors, one or more “general-purpose” microprocessors, one or more special-purpose microprocessors, and/or one or more application specific integrated circuits (ASICS), or some combination thereof. For example, the processor 52 may include one or more reduced instruction set (RISC) processors. The controller 38 may include the memory device 54 that may store information such as control software, look up tables, configuration data, etc.

The memory device 54 may include a tangible, non-transitory, machine-readable medium, such as a volatile memory (e.g., a random-access memory (RAM)) and/or a nonvolatile memory (e.g., a read-only memory (ROM), flash memory, a hard drive, or any other suitable optical, magnetic, or solid-state storage medium, or a combination thereof). The memory device 54 may store a variety of information, which may be suitable for various purposes. For example, the memory device 54 may store machine-readable and/or processor-executable instructions (e.g., firmware or software) for the processor execution.

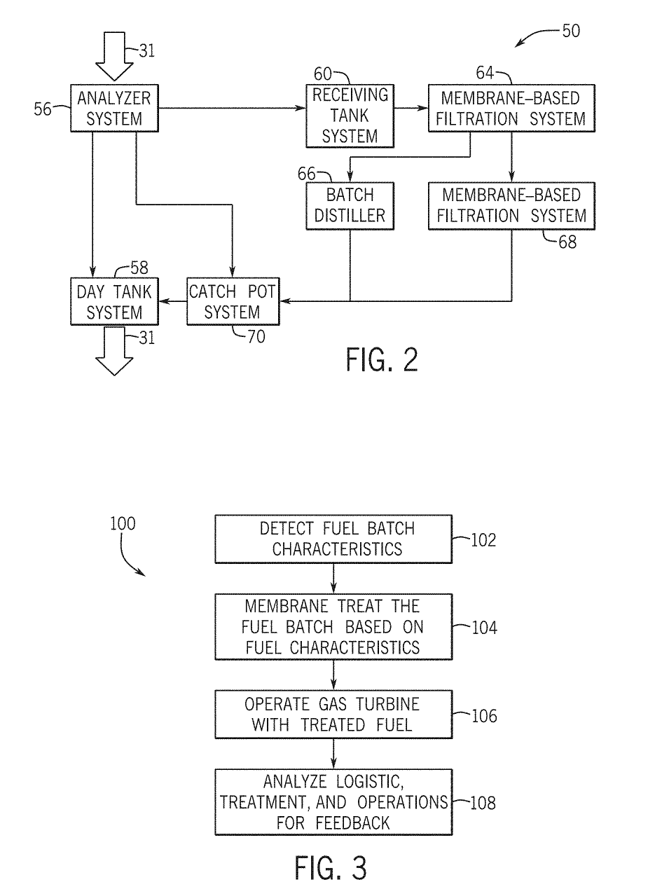

FIG. 2 illustrates an embodiment of a block diagram of the small batch fuel processing system 50. After the fuel 31 is delivered by the transport system 38 to the location of the power production system, at least a portion of the fuel 31 is sent to the small batch fuel processing system 50. A sample is taken from the delivered fuel 31 and analyzed by an analyzer system 56 (e.g., a micro distillation analyzer), which determines certain properties of the fuel 31 (e.g., water content, hydrocarbon content, particulate content, chemical composition, energy content, Wobbe Index, cetane number, octane number, and the like). Fuel 31 that is found to be within the recommended specifications (i.e., the fuel is “in spec”) for use in the gas turbine system 12 may be sent to a day tank system 58 or used for other purposes, described in detail below. In some embodiments, the analyzer system 56 may determine that the delivered fuel 31 is not within the specifications recommended (i.e., the fuel is “out of spec”) for use in the gas turbine system 12. The quality of the fuel for turbines can be brought to turbine specifications by the removal of water, particulate matter, and higher molecular weight hydrocarbons, such as diolefins and vanadium-containing asphaltenes that may cause deposits or corrosion in the turbine hot gas path.

In these embodiments, the delivered fuel 31 may be passed on to a receiving tank system 60. The receiving tank system 60 may be any size and type of container suitable for holding fuel 31. For example, the receiving tank system 60 may store a single shipment (e.g., truck shipment, boat shipment) of fuel 31, or may be large enough to store multiple shipments of fuel 31. After arriving in the receiving tank system 60, the fuel 31 may be either stored and used for other purposes or the fuel 31 may be further refined to be brought within recommended specifications. In some cases, the fuel 31 may be resold, returned to the vendor of the fuel 31, or used for purposes other than combustion in the gas turbine system 12.

Further, an analysis may be performed to calculate the cost of refining the fuel 31 to be in spec, and the increased costs of using the out of spec fuel 31 in the gas turbine system 12 (e.g., the reduced life, lower power output, and reduced efficiency of the gas turbine system 12). If the cost of refining the fuel 31 to be in spec is greater than the increased costs associated with using the out of spec fuel 31, then it may be more cost effective to use the out of spec fuel 31 in the gas turbine system 12 than to refine the out of spec fuel 31 to be in spec. Thus, the out of spec fuel 31 may be utilized in the gas turbine system 12, in some examples.

In other examples, the fuel 31 may be processed through a small batch fuel processing system 62 that may remove some elements from the fuel 31 to bring the fuel 31 within recommended specifications. The small batch fuel processing system 62 may include a membrane-based filtration system 64 for moisture such as water, a batch distiller 66, a membrane-based filtration system 68 for hydrocarbons, or a combination thereof. In some instances, the analyzer system 56 may determine that the fuel 31 contains an amount of water. The fuel 31 may be processed through the membrane-based filtration system 64 to remove some or all of the water from the fuel 31. The membrane-based filtration system 64 includes one or more membranes through which one of the hydrocarbons or the water can pass and the other cannot. It should be appreciated that the permeability characteristics of the membrane-based filtration system 64 may enable water to flow through the membranes (e.g., through a hydrophilic membrane), reject the flow of water (e.g., through a hydrophobic membrane), or a combination thereof. Thus, the membrane-based filtration system 64 separates the water and the hydrocarbons of the fuel 31. In some embodiments, removing the water from the fuel 31 may be sufficient to transform the out of spec fuel 31 into in spec fuel. In these instances, the now in spec fuel 31 may be sent to a catch pot system 70. If removing the water from the fuel 31 is not sufficient to transform the fuel 31 to in spec fuel, then the fuel 31 may be sent to the batch distiller 66, the membrane-based filtration system for hydrocarbons 68, or both.

The batch distiller 66 allows the contents of the fuel 31 to be separated, for example, based on the difference in boiling points between certain chemicals. In order to separate the contents, the batch distiller 66 may include multiple containers and cooling tubes. Further, the batch distiller 66 may operate by applying heat, creating a low-pressure environment, or both. In some embodiments, the fuel 31 is brought to a set of conditions which may cause some compounds within the fuel 31 to boil, while other compounds in the fuel 31 may remain in a liquid state. For example, the batch distiller 66 may be heated to a particular temperature, the pressure may be reduced to a particular pressure, or a combination thereof, to remove some or all of the portion of the fuel 31 that is in spec from the portion of the fuel 31 that is out of spec. Further, the batch distiller 66 may be a distiller that is relatively smaller than distillers commonly used to refine hydrocarbons. For example, the batch distiller 66 may distill only one delivery truck's worth (i.e., less than fifty thousand gallons) of fuel 31. The batch distiller 66 may thus be located at the same location as the power production system 10, thus providing for local fuel 31 distillation.

Further, the membrane-based filtration system 68 may be utilized separate from or in conjunction with the batch distiller 66. The membrane-based filtration system 68 may include one or more membranes that separate hydrocarbons from one another based on weight, molecule size, types of bonds, or viscosity. Thus, the membrane-based filtration system for hydrocarbons 68 may separate the portion of the fuel 31 that is in spec from the portion of the fuel 31 that is out of spec. After the fuel 31 has passed through the batch distiller 66, the membrane-based filtration system 68, or both, the fuel 31 may be sent to the catch pot system 70.

The catch pot system 70 may be utilized to store the fuel 31 for further testing or for use in the gas turbine system 12. For example, if the delivered fuel 31 is not within the recommended specifications, the fuel 31 may be sent to the catch pot system 70 to be mixed with other fuel 31 that is within the recommended specifications so that all of the fuel 31 in the catch pot system 70 is within the recommended specifications. In other embodiments, the catch pot system 70 may receive the fuel 31 that has passed through the small batch fuel processing system 50. The fuel 31 that has passed through the small batch fuel processing system 50 may still be out of spec, but may be closer to in spec than it was before passing through the small batch fuel processing system 50. In these instances, the fuel 31 that has passed through the small batch fuel processing system 50, but is still out of spec may be mixed with fuel 31 that is in spec so that the combination of the in spec and out of spec fuel 31 is in spec. After fuel 31 has arrived in the catch pot system 70, the analyzer system 56 may be utilized to test the characteristics of the fuel 31. If the fuel 31 is still out of spec, the fuel 31 may be sent back to the receiving tank system 60, or utilized for other purposes (e.g., resold, returned to a vendor, used for purposes other than combustion in the gas turbine system 12).

If the analyzer system 56 determines that the fuel 31 is in spec, then the fuel 31 may be sent to the day tank system 58 or to the gas turbine system 12. The day tank system 58 may be a container that is capable of holding enough fuel 31 as may be utilized by the power production system 10 in a portion of or multiple days. Further, the day tank system 58 may include multiple containers and the fuel 31 may be sent to different containers depending on certain characteristics of the fuel 31 (e.g., the quality). After fuel 31 has arrived in the day tank system 58, the fuel 31 may be sent to the appropriate systems to be utilized in the combustion system 22 of the gas turbine system 12, as described above.

FIG. 3 depicts a process 100 for analyzing the fuel 31 and improving the quality of the fuel 31 for use in the gas turbine system 12. Although the process 100 describes a number of operations that may be performed, it should be noted that the process 100 may be performed in a variety of suitable orders. All of the operations of the process 100 may not be performed. The process 100 may be implemented as computer code or instructions stored in the memory 54 and executable by the processor 52.

After the fuel 31 arrives at the location of the power production system 10, the fuel 31 is run through the analyzer system 56 for detection (block 102) of the fuel 31 characteristics. As discussed above, the analyzer system 56 may determine which hydrocarbons are present and in which percentage, water content, hydrocarbon content, particulate content, chemical composition, energy content, Wobbe Index, cetane number, octane number, and the like. Further, the analyzer system 56 may determine the water content of the fuel 31 and whether there are any impurities contained within the fuel 31. If the analyzer system 56 determines that the fuel 31 is not within the recommended specifications, the fuel 31 may be processed further so that it conforms to the recommended specifications.

For example, the fuel 31 may undergo membrane treatment (block 104) based on the characteristics determined by the analyzer system 56. As described above, the fuel 31 may be sent through the membrane-based filtration system 64 to separate water from the fuel 31. In some embodiments, the fuel 31 may be sent through the batch distiller 56, the membrane-based filtration system 68, or a combination thereof, to separate the portions of the fuel 31 that are in spec from the portions of the fuel 31 that are out of spec.

After the fuel 31 has undergone distillation (block 104) treatment, the now treated fuel 31 may be used to operate (block 106) the gas turbine system 12. As discussed above, the fuel 31 may be received by the day tank system 58, where it may be utilized by the gas turbine system 12 (e.g., by the combustion system 22).

Next, the process 100 may analyze (block 108) the logistics of the fuel 31 delivery for feedback. In particular, the process 100 may track the characteristics of the fuel 31 as measured by the analyzer system 56. For example, the process 100 may analyze the quality of each shipment of fuel 31 to determine statistics such as percentage of fuel 31 delivered within the recommended specification, average quality of the fuel 31, and relate the fuel 31 characteristics to each supplier. This allows the process 100 to track the fuel 31 supplied from each supplier.

Further, the process 100 may analyze (block 108) the treatment of the fuel 31 for feedback. In particular, the process 100 may track the change in fuel 31 properties before and after going through the distillation (block 104) treatment. For example, the process 100 may track the time and cost of improving the properties of the fuel 31 to determine the value in treating the fuel 31, as well as the improvement measured in Wobbe units, percent hydrocarbons, moisture removal percent, and so on. Specifically, the process 100 may compare the time and cost of utilizing the distillation process to the time and cost of purchasing a new batch of fuel 31 that is within the recommended specifications. After the fuel 31 has undergone distillation (block 104) treatment, the now treated fuel 31 may be used to operate (block 106) the gas turbine system 12. As discussed above, the fuel 31 may be received by the day tank system 58, where it may be utilized by the gas turbine system 12 (e.g., by the combustion system 22).

Next, the process 100 may analyze (block 108) the logistics of the fuel 31 delivery for feedback. In particular, the process 100 may track the characteristics of the fuel 31 as measured by the analyzer system 56. For example, the process 100 may analyze the quality of each shipment of fuel 31 to determine statistics such as percentage of fuel 31 delivered within the recommended specification, average quality of the fuel 31, and link (e.g., via a database or similar system) the fuel 31 characteristics to each supplier. This allows the process 100 to log and historically analyze the fuel 31 supplied from each supplier.

Further, the process 100 may analyze (block 108) the treatment of the fuel 31 for feedback. In particular, the process 100 may track the change in fuel 31 quality before and after going through the distillation (block 104) treatment. For example, the process 100 may track the time and cost of improving the quality of the fuel 31 to determine the value in treating the fuel 31. Specifically, the process 100 may compare the time and cost of utilizing the distillation process to the time and cost of purchasing a new batch of fuel 31 that is within the recommended specifications.

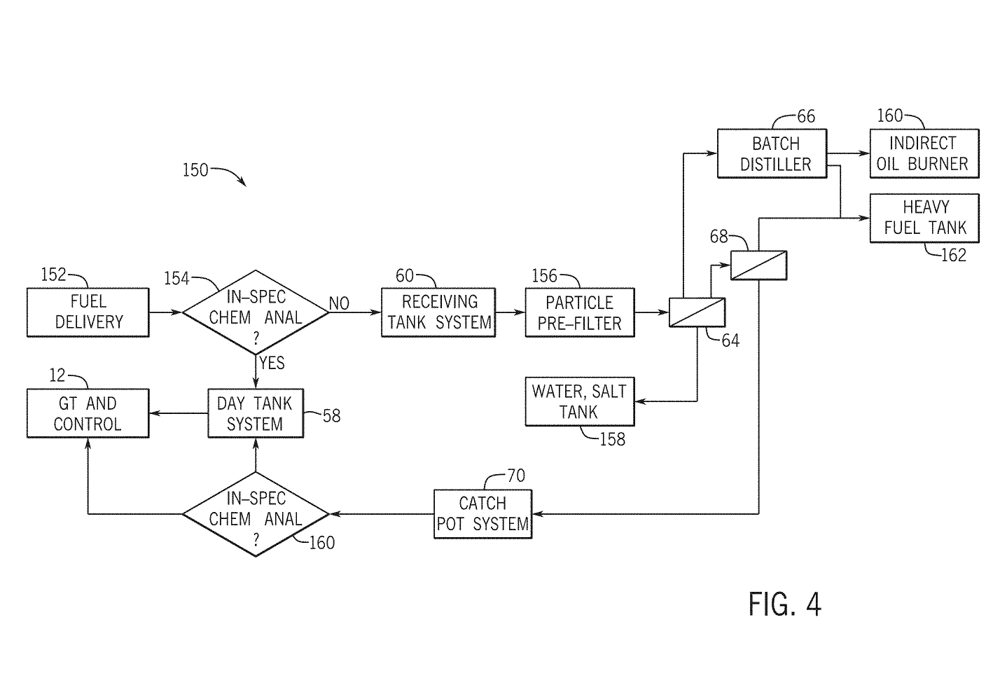

FIG. 4 is a block diagram of a flow chart 150 that may be utilized to improve the quality of the fuel 31 after it arrives at the location of the gas production system 10. The fuel 31 is brought to the location of the gas production system 10 by a delivery vehicle 152 (e.g., a truck or a boat). Then, the fuel 31 undergoes a chemical analysis (block 154) to determine whether the fuel 31 is within the recommended specifications for use in the gas turbine system 12. As discussed above, the chemical analysis may be performed by the analyzer system 56 to determine the characteristics of the fuel 31. If the fuel 31 is found to be within the recommended specifications (i.e., “in spec”), the fuel 31 is sent to the day tank system 58, where it may be utilized in the gas turbine system 12. If the fuel 31 is found to be outside of the recommended specifications (i.e., “out of spec”), the fuel 31 is sent to the receiving tank system 60.

Fuel 31 that is sent to the receiving tank system 60 may be processed in various ways. In some embodiments, a second analysis may be performed to determine the optimal way of dealing with the fuel 31 that is not within the recommended specifications. In some instances, it may be cost effective to further improve the quality of the fuel 31 so that it may be brought within the recommended specifications. In other instances, it may be cost effective to send the fuel 31 to another location. For example, the fuel 31 may be returned to the supplier or sold to a buyer.

In instances where it is determined that the fuel 31 may be brought within the recommended specifications, there are multiple options for improving the quality of the fuel 31. In some embodiments, the out of spec fuel 31 may be combined with in spec fuel 31 so that the combination of the two fuels produces in spec fuel 31. In other instances, it may be more cost effective to improve the out of spec fuel 31 by refining the fuel 31.

Before sending the fuel 31 to the membrane-based filtration for water 64, the fuel 31 may be sent through a particle pre-filter 156 to remove particulate matter from the fuel 31. The particle pre-filter 156 may be a filter that removes particles from a fluid (i.e., the fuel 31) by physically blocking the particles as the fuel 31 flows through the particle pre-filter 156. Utilizing the particle pre-filter 156 may increase the efficiency, life span, or both of the membrane-based filtration for water 64 or the membrane-based filtration for hydrocarbons 68.

If is it determined that the fuel 31 contains water that should be removed, the fuel 31 may be sent through the membrane-based filtration for water 64. As discussed above, the membrane-based filtration for water 64 includes one or more membranes through which one of the hydrocarbons or the water can pass and the other cannot. Thus, the membrane-based filtration for water 64 separates the water and the hydrocarbons of the fuel 31. In some embodiments, removing the water from the fuel 31 may be sufficient to bring the out of spec fuel 31 in spec. In these instances, the fuel 31 may be sent to the catch pot system 70. If removing the water from the fuel 31 is not sufficient to bring the fuel 31 in spec, then the fuel 31 may be sent to the batch distiller 66, the membrane-based filtration for hydrocarbons 68, or both. Further, the water that is filtered out by the membrane-based filtration for water 64 may be collected in a water, salt tank 158. The water collected in the water, salt tank 158 may be analyzed to determine the contents of the water (e.g., presence of solutes, determining which solutes are present and in what percentage). In some embodiments, the water in the water, salt tank 158 may be used to provide cooling in other parts of the power production system 10. In other embodiments, the water may be disposed of in a proper manner.

The batch distiller 66 allows the contents of the fuel 31 to be separated based on the difference in boiling points. In order to do so, the batch distiller 66 may include multiple containers and cooling tubes. Further, the batch distiller 66 may operate by applying heat, creating a low-pressure environment, or both. In some embodiments, the fuel 31 is brought to a set of conditions which may cause some compounds within the fuel to boil, while other compounds in the fuel 31 may remain in a liquid state. In particular, the portion of the fuel 31 that is in spec may have a lower boiling point that the portion of the fuel 31 that is out of spec. This allows the batch distiller 66 to separate the out of spec fuel 31 from the in spec fuel 31 by boiling the in spec fuel 31 while the out of spec fuel 31 remains in a liquid state. For example, the batch distiller 66 may be heated to a particular temperature, the pressure may be reduced to a particular pressure, or a combination thereof, to remove some or all of the portion of the fuel 31 that is in spec from the portion of the fuel 31 that is out of spec. Further, the batch distiller 66 may be a distiller that is relatively smaller than distillers commonly used to refine hydrocarbons. For example, the batch distiller 66 may distill only one delivery truck's worth (i.e., less than fifty thousand gallons) of fuel 31.

Further, the membrane-based filtration for hydrocarbons 68 may be utilized separate from or in conjunction with the batch distiller 66. The membrane-based filtration for hydrocarbons 68 may include one or more membranes that separate hydrocarbons from one another based on molecular weight, molecule size, types of bonds, or viscosity. Thus, the membrane-based filtration for hydrocarbons 68 may separate the portion of the fuel 31 that is in spec from the portion of the fuel 31 that is out of spec. After the fuel 31 has passed through the batch distiller 66, the membrane-based filtration for hydrocarbons 68, or both, the fuel 31 may be sent to the catch pot system 70.

The out of spec fuel 31 that has been separated from the in spec fuel 31 may be recovered and used for other purposes. For example, the out of spec fuel 31 may be sent to an indirect oil burner 160 where the out of spec fuel 31 may be burned to provide heat for the distilling process in the batch distiller 66. In some embodiments, the indirect oil burner 160 may not be utilized, or the collected amount of out of spec fuel 31 may exceed the amount used by the indirect oil burner 160. In these instances, the out of spec fuel 31 may be collected in the heavy fuel tank 162 where the out of spec fuel 31 may be sold to a buyer or disposed of.

The fuel 31 that is sent to the catch pot system 70 may be chemically analyzed (block 160) to determine the characteristics of the fuel 31. If the fuel 31 in the catch pot system 70 is found to still be out of spec, the fuel 31 may be sent back to the receiving tank system 60. If the fuel 31 is found to be in spec, then the fuel 31 may be sent to the day tank system 58, or the catch pot system 70 may be utilized for the same purpose as the day tank system 58. That is, the catch pot system 70 may send the fuel 31 to the gas turbine system 12.

This written description uses examples to disclose the subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function]. . . ” or “step for [perform]ing [a function]. . . ”, it is intended that such elements are to be interpreted under 35 U.S.C. 112(f). However, for any claims containing elements designated in any other manner, it is intended that such elements are not to be interpreted under 35 U.S.C. 112(f). This written description uses examples to disclose the subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A system comprising:
a gas turbine system configured to combust a fuel to produce a power, wherein the gas turbine system is disposed in a gas turbine site;
an analyzer system configured to determine a plurality of batch fuel characteristics of a batch of the fuel, wherein the batch of the fuel is delivered via a transport system; and
a small batch fuel processing system configured to receive the batch of fuel and filter the batch of fuel through a membrane-based system to a filtered batch of fuel based on the plurality of fuel characteristics, wherein the filtered batch of fuel adheres to manufacturer fuel recommended characteristics for use in the gas turbine system.

2. The system of claim 1, comprising a batch fuel receiving tank system, wherein the batch fuel receiving tank system is configured to receive the batch of fuel based on a derivation of the plurality of batch fuel characteristics determining that the batch of fuel is not suitable for use by the gas turbine system.

3. The system of claim 1, comprising a day tank system, wherein the day tank system is configured to receive the batch of fuel based on a derivation of the plurality of batch fuel characteristics determining that the batch of fuel is suitable for use by the gas turbine system.

4. The system of claim 1, wherein the small batch fuel processing system is disposed in the gas turbine site.

5. The system of claim 1, wherein the membrane-based system comprises a membrane-based filtration for water configured to filter water from the batch of fuel to produce a water-filtered batch of fuel.

6. The system of claim 5, comprising a membrane-based filtration for hydrocarbons configured to receive the water-filtered batch of fuel and filter the water-filtered batch of fuel to produce the filtered batch of fuel and a heavy fuel filtered batch of fuel, wherein the heavy fuel filtered batch of fuel does not adhere to manufacturer fuel recommended characteristics for use in the gas turbine system.

7. The system of claim 5, comprising a small batch fuel distiller system configured to receive the water-filtered batch of fuel and distill the water-filtered batch of fuel to produce the filtered batch of fuel and a heavy fuel filtered batch of fuel, wherein the heavy fuel filtered batch of fuel does not adhere to manufacturer fuel recommended characteristics for use in the gas turbine system.

8. The system of claim 1, comprising a heavy fuel tank system, wherein the heavy fuel tank system is configured to receive a heavy fuel filtered batch of fuel, wherein the heavy fuel filtered batch of fuel comprises a portion of the batch of fuel and the heavy fuel filtered batch of fuel does not adhere to manufacturer fuel recommended characteristics for use in the gas turbine system.

9. The system of claim 1, comprising the transport system, wherein the transport system comprises a vehicle, a fluid conduit transport system, or a combination thereof.

10. A method, comprising:
receiving, via a transport system, a batch of fuel;
analyzing, via an analyzer system, the batch of fuel to derive a plurality of batch fuel characteristics;
determining, via a processor, a suitability of the batch of fuel to be used in a gas turbine system based on the plurality of batch fuel characteristics; and
filtering, via a small batch fuel processing system, the batch of fuel to produce a filtered batch of fuel that adheres to manufacturer fuel recommended criteria for use in the gas turbine system when the batch of fuel is determined to be not suitable for use in the gas turbine system.

11. The method of claim 10, comprising depositing the batch of fuel into a batch fuel receiving tank system before filtering when the batch of fuel is determined to be not suitable for use in the gas turbine system.

12. The method of claim 10, wherein filtering comprises passing the batch of fuel through a membrane configured to separate a first portion of the batch of fuel from a second portion of the batch of fuel based on a difference in molecular characteristics, and wherein the first portion of the batch of fuel adheres to manufacturer fuel recommended criteria for use in the gas turbine system.

13. The method of claim 10, comprising pre-filtering, via a particle pre-filter system, the batch of fuel, wherein pre-filtering removes a plurality of particles from the batch of fuel based on a size of the plurality of particles.

14. The method of claim 10, wherein the transport system comprises a vehicle, a fluid conduit transport system, or a combination thereof

15. The method of claim 10, comprising receiving, via a catch pot system, the filtered batch of fuel and analyzing, via the analyzer system, the filtered batch of fuel to derive a plurality of filtered batch fuel characteristics.

16. One or more tangible, non-transitory, machine-readable media comprising instructions configured to cause a processor to:
receive, via a transport system, a batch of fuel;
analyze, via an analyzer system, the batch of fuel to derive a plurality of batch fuel characteristics;
determine a suitability of the batch of fuel to be used in a gas turbine system based on the plurality of batch fuel characteristics; and
filter, via a small batch fuel processing system, the batch of fuel to produce a filtered batch of fuel that adheres to manufacturer fuel recommended criteria for use in the gas turbine system when the batch of fuel is determined to be not suitable for use in the gas turbine system.

17. The one or more tangible, non-transitory, machine-readable media comprising instructions of claim 16, configured to cause the processor to deposit the batch of fuel into a batch fuel receiving tank system before filtering when the batch of fuel is determined to be not suitable for use in the gas turbine system.

18. The one or more tangible, non-transitory, machine-readable media comprising instructions of claim 16, wherein filtering comprises passing the batch of fuel through a membrane configured to separate a first portion of the batch of fuel from a second portion of the batch of fuel based on a difference in molecular characteristics, and wherein the first portion of the batch of fuel adheres to manufacturer fuel recommended criteria for use in the gas turbine system.

19. The one or more tangible, non-transitory, machine-readable media comprising instructions of claim 16, configured to cause the processor to pre-filter, via a particle pre-filter system, the batch of fuel, wherein pre-filtering removes a plurality of particles from the batch of fuel based on a size of the plurality of particles.

20. The one or more tangible, non-transitory, machine-readable media comprising instructions of claim 16, configured to cause the processor to receive, via a catch pot system, the filtered batch of fuel and analyze, via the analyzer system, the filtered batch of fuel to derive a plurality of filtered batch fuel characteristics.
